(19) United States
(12) Patent Application Publication
(10) Pub. No.: US 2001/0049514 A1
(43) Pub. Date: Dec. 6, 2001

(54) ABSORBENT COMPOSITES WITH ENHANCED INTAKE PROPERTIES

(76) Inventors: RICHARD NORRIS DODGE II, APPLETON, WI (US); SRIDHAR RANGANATHAN, SUWANEE, GA (US); SANDRA MARIE YARBROUGH, MENASHA, WI (US); WENDY LYNN VAN DYKE, APPLETON, WI (US); MICHAEL JOHN NIEMEYER, APPLETON, WI (US); YONG LI, APPLETON, WI (US)

Correspondence Address: JOHN S. PRATT, KILPATRICK STOCKTON LLP, 1100 PEACHTREE, SUITE 2800, ATLANTA, GA, 30309, US

(21) Appl. No.: 09/475,829

(22) Filed: Dec. 30, 1999

Related U.S. Application Data
(60) Provisional application No. 60/114,433, filed on Dec. 31, 1998.

(51) Int. Cl. A61F 13/15; A61F 13/20; B32B 5/16; B32B 9/00; B32B 15/02; B32B 17/02; B32B 19/00; B32B 21/02; B32B 23/02; B32B 27/02

(52) U.S. Cl. 604/368; 604/370; 604/372; 428/402

(57) ABSTRACT

The present invention is directed to absorbent composites having enhanced intake properties. The absorbent composites of the present invention have a Composite Permeability (CP) value at full swelling of greater than about 100 ×10⁻⁸ cm². Further, the absorbent composites, of the present invention have a Composite Permeability/3^{rd} Insult Fluid Intake Flowback Evaluation (FIFE) intake relationship, which results in enhanced intake properties. The present invention is also directed to a method of making absorbent composites having enhanced intake properties. The present invention is further directed to absorbent composites and their applicability in disposable personal care products.

### CROSS-REFERENCE TO RELATED APPLICATIONS

This application claims the benefit of U.S. Provisional Application Serial No. 60/114,433, filed on Dec. 31, 1998 and assigned to Kimberly-Clark Worldwide, Inc.

### FIELD OF THE INVENTION

The present invention is directed to absorbent composites having enhanced intake properties. The present invention is also directed to a method of making absorbent composites having enhanced intake properties. The present invention is further directed to absorbent composites and their applicability in disposable personal care products.

### BACKGROUND OF THE INVENTION

In the manufacture of disposable cliapers, there is continual effort to improve the performance characteristics of the diaper. Although the structure of a diaper has many components, in many instances the in-use performance of the diaper is directly related to the characteristics of the absorbent composite contained within the diaper. Accordingly, diaper manufacturers strive to find ways of improving the properties of the absorbent composite, including in-use absorbency, in order to reduce the tendency of the diaper to leak.

One means of reducing the leakage of a diaper has been the extensive use of superabsorbent materials. Recent trends in commercial diaper design have been to use more superabsorbent materials and less fiber in order to make the diaper thinner. However, notwithstanding the increase in total absorbent capacity contributed by the addition of larger amounts of superabsorbent material, such diapers often still suffer from excessive leaking during use.

One reason that diapers with a high content of superabsorbent materials still leak is that many absorbent materials are unable to absorb a liquid at the rate at which the liquid is applied to the absorbent composite during use. The addition of fibrous material to the absorbent composite improves the leakage of an absorbent composite by temporarily holding the liquid until the superabsorbent material absorbs it. Fibers also serve to separate the particles of superabsorbent material so that gel-blocking does not occur. As used herein, the term “gel-blocking” refers to the situation wherein particles of superabsorbent material deform during swelling and block the interstitial spaces between the particles, or between the particles and the fibers, thus preventing the flow of liquid through the interstitial spaces. Even when fibrous material is incorporated into an absorbent composite, a poor choice of a superabsorbent material, especially one which exhibits gel-blocking behavior within the absorbent composite, results in poor liquid handling properties initially and later in the life cycle of the absorbent composite. Consequently, the choice of absorbent composite materials greatly affects the in-use absorbency and leakage of the absorbent product.

Another problem with commercially available diapers is the tendency of diapers to leak after multiple insults. As used herein, the term “insults” refers to a single introduction of liquid into the absorbent composite or diaper. During use, a diaper is typically exposed to multiple insults during the life cycle of the diaper. To reduce diaper leakage during the life cycle of the diaper, it is desirable to maintain the level of intake performance of the absorbent composite throughout the life of the product.

A number of U.S. patents address different problems associated with absorbent composites. For example, U.S. Pat. No. 5,304,161 issued to Noel and Ahr teaches the use of a multi-layer absorbent structure in which an upper acquisition/distribution layer exhibits a rapid liquid acquisition rate. U.S. Pat. No.5,047,023 issued to Berg teaches the benefits of an acquisition zone having a low density and low basis weight to allow rapid intake of liquid. U.S. Pat. No. 5,348,547 issued to Payne et al. teaches a dual-layered absorbent system in which an acquisition layer consists of a low density region and a high density region to allow rapid intake and subsequent distribution of liquid from an insult point. International patent publication number WO 98/29071 to Molnlycke AB describes an improved acquisition/transport layer that has improved tactile characteristics, which allows it to be used directly in contact with a wearer's skin. U.S. Pat. No. 5,397,316 to LaVon et al. describes various configuations of polymeric foams, which have been designed to rapidly acquire liquid.

The aforementioned patents disclose specific absorbent composite properties, which result in improved composite performance. In general, the aforementioned patents and publications teach various configurations of absorbent composite materials to enhance fluid intake rate However, the aforementioned patents do not specifically address the problems mentioned above, namely, improving leakage/intake over the life cycle of the absorbent composite.

What is needed in the art is an absorbent composite having optimum composite properties. What is also needed in the art is an absorbent composite, which exhibits improved fluid intake rate, and superior fluid intake of multiple insults over the life of the composite, without the problems associated with known absorbent composites.

### SUMMARY OF THE INVENTION

The present invention is directed to absorbent composites, which have been developed to address the above-described problems associated with currently available, absorbent composites and other absorbent composites described in literature. The absorbent composites of the present invention have improved composite intake properties as a result of having a Composite Permeability value at full swelling of greater than about 100×10⁻⁸ cm² and a Composite Permeability value/3^{rd} Insult Fluid Intake Flowback Evaluation (FIFE) intake rate relationship, wherein the Composite Permeability value varies depending on the 3^{rd} Insult FIFE intake rate. This combination of composite properties is an indication of an absorbent composite's ability to have improved fluid intake rate, and superior fluid intake of multiple insults over the life of the composite. Unlike known absorbent composites, which lose their fluid intake performance over the life of the composite, the absorbent composites of the present invention perform exceptionally well, exhibiting superior fluid intake after multiple insults to the composite.

The present invention is also directed to a method of making absorbent composites having a Composite Permeability value at full swelling of greater than about 100×10⁻⁸ cm² and a Composite Permeability value/3^{rd} Insult Fluid Intake Flowback Evaluation (FIFE) intake rate relationship, wherein the Composite Permeability value varies depending on the 3^{rd} Insult FIFE intake rate. The absorbent composites of the present invention may be made by a variety of processes.

The present invention is further directed to absorbent composites comprising fibrous material, and their applicability in disposable personal care products. The absorbent composites of the present invention are particularly useful as absorbent components in personal care products such as diapers, feminine pads, panty liners, incontinence products, and training pants.

### BRIEF DESCRIPTION OF THE FIGURES

FIGS. 1a-c are an illustration of equipment for determining the Composite Permeability value of an absorbent composite.

FIG. 2 is an illustration of equipment for determining the Fluid Intake Flowback Evaluation (FIFE) value of an absorbent composite.

FIG. 3 is an illustration of equipment for determining the Intake/Desorption value of an absorbent composite.

### DESCRIPTION OF THE PREFERRED EMBODIMENTS

The present invention is directed to absorbent composites, wherein the absorbent composites possess the ability to maintain exceptional intake performance even after multiple insults to the composite. The present invention achieves these results by approaching the problems of intake performance and leakage in an unconventional manner. Traditionally, the approach taken to address fluid intake has been to strategically locate relatively large amounts of superabsorbents and/or superabsorbents having a high capacity under load in an absorbent composite. The goal was to produce an absorbent composite having increased capacity to ultimately provide to the composite improved fluid intake performance. However, it has been determined that the pursuit of higher superabsorbent capacity inevitably leads to limited performance improvement. In particular, this approach results in a decrease of the intake performance of the composite over the life cycle of the composite. The present invention has discovered that methods for achieving rapid liquid uptake, as well as, enhanced intake performance over the life of the composite can be achieved with relatively high amounts of superabsorbent materials by concentrating on the composite permeability and its relationship to the 3^{rd} Insult Fluid Intake Flowback Evaluation (FIFE) intake rate of the composite.

The absorbent composites of the present invention desirably possess constant or improved fluid intake over the life of the composite. The fundamental absorbent property of composite permeability of an absorbent material is a key to fast intake. One method of measuring composite permeability is with the composite Permeability test, which is described in detail below. This test measures the time required for a fixed volume of liquid to flow through a pre-saturated composite in the z-direction. As shown in Table 1, the proper choice of absorbent composite materials, including fibers and/or superabsorbent material, enable the formation of an absorbent composite having a composite permeability of greater than about 150×10⁻⁸ cm². As shown in Table 1, all of the first five absorbent composites (Samples 1-5) exhibit a high composite permeability (>150×10⁻⁸ cm²), However, some combinations of absorbent composite materials result in absorbent composites having a composite permeability of much less than 150×10⁻⁸ cm², as shown by Samples 6 and 7.

TABLE 1
Composite Permeability for Absorbent Composites
Containing 50 wt % Fibers and 50 wt % SAM

| | | SAM | | Composite |
|---|---|---|---|---|
| Composite | Fibrous | Desig- | Superabsorbent | Permeability |
| Sample | Material | nation | Material | (×10⁻⁸ cm²) |
| 1 | Wood Pulp | S1 | Stockhausen | ˜191 |
| | | | W-65431 | |
| 2 | Wood Pulp | D2 | Dow AFA-173- | ˜177 |
| | | | 60B | |
| 3 | Wood Pulp | D3 | Dow XUS | ˜202 |
| | | | 40665.07 | |
| 4 | Wood Pulp | D4 | Dow XU | ˜192 |
| | | | 40671.00 | |
| 5 | Wood Pulp | D6 | Dow XUS | ˜168 |
| | | | 40667.01 | |
| 6 | Wood Pulp | — | Stockhausen | ˜112 |
| | | | Favor 880 | |
| 7 | Wood Pulp | — | Dow DryTech | ˜61 |
| | | | 2035 | |

Another important measure of composite intake performance is measured by the Fluid Intake Flowback Evaluation (FIFE) test, which is described in detail below. The FIFE test measures how fast liquid can flow into a material. Table 2 shows the 3^{rd} insult FIFE intake rates for a variety of absorbent composites containing 50 wt % superabsorbent material and 50 wt % fibers. It can be seen that absorbent composites containing different superabsorbents exhibit different FIFE intake rates. As shown in Table 2, all of the first five absorbent composites (Samples 1-5) exhibit fast intake rates (>2.75 ml/sec). However, some combinations of absorbent composite materials result in absorbent composites having an intake rate of much less than 2.75 ml/sec, as shown by Samples 6 and 7.

TABLE 2
3^{rd} Insult FIFE Rate for Absorbent Composites
Containing 50 wt % Fibers and 50 wt % SAM

| | | SAM | | 3^{rd} Insult |
|---|---|---|---|---|
| Composite | Fibrous | Desig- | Superabsorbent | FIFE Rate |
| Sample | Material | nation | Material | (ml/sec) |
| 1 | Wood Pulp | S1 | Stockhausen | ˜3.2 |
| | | | W-65431 | |
| 2 | Wood Pulp | D2 | Dow AFA-173- | ˜3.1 |
| | | | 60B | |
| 3 | Wood Pulp | D3 | Dow XUS | ˜3.1 |
| | | | 40665.07 | |
| 4 | Wood Pulp | D4 | Dow XU | ˜3.4 |
| | | | 40671.00 | |
| 5 | Wood Pulp | D6 | Dow XUS | ˜3.0 |
| | | | 40667.01 | |
| 6 | Wood Pulp | — | Stockhausen | ˜2.1 |
| | | | Favor 880 | |
| 7 | Wood Pulp | — | Dow DryTech | ˜1.6 |
| | | | 2035 | |

The improved intake behavior as seen by the 3^{rd} Insult FIFE Intake Rate may be controlled by the amount of superabsorbent material present in the absorbent composite. Table 3 shows the 3^{rd} Insult FIFE Intake Rate for two sets of composites containing one of two superabsorbent materials (identified as Stockhausen Favor 880 and Dow XUS 40665.07). For each type of superabsorbent material, composites containing either 30, 40, 50, or 60 wt % superabsorbent material were prepared and evaluated. All composites had a total basis weight of 400 gsm. This results in composites having a superabsorbent basis weight of 120, 160, 200, or 240 gsm.

TABLE 3
3^{rd} Insult FIFE Rate for Absorbent Composites with
Variable SAM Weight Percent

| | | | | 3^{rd} Insult FIFE Rate | |
|---|---|---|---|---|---|
| | | | | (ml/sec) | |
| | | | SAM Basis | | Dow XUS |
| Composite | Fibrous | SAM | Weight | Favor | 40665.07 |
| Sample | Material | wt % | (gsm) | 880 | (D3) |
| 1 | Wood Pulp | 30 | 120 | 6.6 | 6.6 |
| 2 | Wood Pulp | 40 | 160 | 4 | 5.7 |
| 3 | Wood Pulp | 50 | 200 | 2.2 | 3.1 |
| 4 | Wood Pulp | 60 | 240 | 2 | 3 |

As can be seen in Table 3, as the amount of superabsorbent material in the composite changes, the 3^{rd} Insult FIFE Intake Rate of the composite changes. In addition, at 40, 50, and 60 wt % superabsorbent levels, a composite comprising Dow XUS 40665.07 superabsorbent material exhibits a faster, more desirable, 3^{rd} Insult FIFE Intake Rate compared to a composite comprising Stockhausen Favor 880 superabsorbent material.

To further demonstrate the impact of the type and amount of superabsorbent material present in the composite on the composite intake behavior, Table 4 shows the 3^{rd} Insult FIFE Intake Rate for two sets of absorbent composites containing one of two superabsorbent materials (identified as Stockhausen Favor 880 and Dow XUS 40665.07). However, in these two sets, for each type of superabsorbent material, composites having a total composite basis weight of either 200, 300, 400, or 500 gsm were prepared and evaluated. All composites had 50 wt % wood pulp fibers and 50 wt % superabsorbent material. This results in absorbent composites having a superabsorbent basis weight of 100, 150, 200, or 250 gsm.

TABLE 4
3^{rd} Insult FIFE Rate for Absorbent Composites with
Variable Composite Basis Weight

| | | | | 3^{rd} Insult FIFE | |
|---|---|---|---|---|---|
| | | Composite | | Rate (ml/sec) | |
| | | Basis | SAM Basis | | Dow XUS |
| Composite | Fibrous | Weight | Weight | Favor | 40665.07 |
| Sample | Material | (gsm) | (gsm) | 880 | (D3) |
| 1 | Wood Pulp | 200 | 100 | 4.4 | 4.4 |
| 2 | Wood Pulp | 300 | 150 | 3.3 | 5.5 |
| 3 | Wood Pulp | 400 | 200 | 2.2 | 3.3 |
| 4 | Wood Pulp | 500 | 250 | 2.5 | 3.5 |

As can be seen in Table 4, as the composite basis weight (and superabsorbent basis weight) changes, the 3^{rd} Insult FIFE Intake Rate of the composite changes. In addition, at superabsorbent basis weights of 150, 200, or 250 gsm, a composite comprising Dow XUS 40665.07 superabsorbent material exhibits a faster, more desirable, 3^{rd} Insult FIFE Intake Rate compared to a composite comprising Stockhausen Favor 880 superabsorbent material.

As would be expected, the composite permeability may also be controlled by the amount of superabsorbent material present in the absorbent composite. Table 5 shows the composite permeability for two sets of composites containing one of two superabsorbent materials (identified as Stockhausen Favor 880 and Dow XUS 40665.07). For each type of superabsorbent material, composites containing either 30, 40, 50, or 60 wt % superabsorbent material were prepared and evaluated. All composites had a total basis weight of 400 gsm. This results in composites having a superabsorbent basis weight of 120, 160, 200, or 240 gsm.

TABLE 5
Composite Permeability for Absorbent Composites with
Variable SAM Weight Percent

| | | | | Composite Permeability | |
|---|---|---|---|---|---|
| | | | | (×10⁻⁸ cm²) | |
| | | | SAM Basis | | Dow XUS |
| Composite | Fibrous | SAM | Weight | Favor | 40665.07 |
| Sample | Material | wt % | (gsm) | 880 | (D3) |
| 1 | Wood Pulp | 30 | 120 | 161 | 226 |
| 2 | Wood Pulp | 40 | 160 | 172 | 255 |
| 3 | Wood Pulp | 50 | 200 | 112 | 202 |
| 4 | Wood Pulp | 60 | 240 | 63 | 159 |

As can be seen in Table 5, as the amount of superabsorbent material in the composite changes, the composite permeability of the composite changes. In addition, at 30, 40, 50, and 60 wt % superabsorbent levels, a composite comprising Dow XUS 40665.07 superabsorbent material exhibits a higher composite permeability compared to a composite comprising Stockhausen Favor 880 superabsorbent material.

The absorbent composites of the present invention have a Composite Permeability value at full swelling of greater than about 100×10⁻⁸ cm². Desirably, the absorbent composites of the present invention have a CP value at full swelling of greater than about 175×10⁻⁸ cm². More desirably, the absorbent composites of the present invention have a CP value at full swelling of greater than about 190×10⁻⁸ cm². Even more desirably, the absorbent composites of the present invention have a CP value at full swelling of greater than about 205×10⁻⁸ cm². Most desirably, the absorbent composites of the present invention have a CP value at full swelling of greater than about 225×10⁻⁸ cm². When the absorbent composites of the present invention have a CP value at full swelling of greater than about 175×10⁻⁸ cm², desirably the 3^{rd} Insult FIFE intake rate is greater than about 2.00 ml/sec. More desirably, when the absorbent composites of the present invention have a CP value at full swelling of greater than about 175×10⁻⁸ cm², the 3^{rd} Insult FIFE intake rate is greater than about 2.50 ml/sec. Even more desirably, when the absorbent composites of the present invention have a CP value at full swelling of greater than about 175×10⁻⁸ cm², the 3^{rd} Insult FIFE intake rate is greater than about 2.75 ml/sec. Most desirably, when the absorbent composites of the present invention have a CP value at full swelling of greater than about 175×10⁻⁸ cm², the 3^{rd} Insult FIFE intake rate is greater than about 3.00 ml/sec.

In one embodiment, the absorbent composites of the present invention have a Composite Permeability value/3^{rd} Insult Fluid Intake Flowback Evaluation (FIFE) intake rate relationship, wherein the Composite Permeability value varies depending on the 3^{rd} Insult FIFE intake rate. Desirably, when the 3^{rd} Insult FIFE intake rate (IR) of the absorbent composite is greater than 0 ml/sec and less than about 3.00 ml/sec, the Composite Permeability (CP) value at full swelling of the absorbent composite is given by the following equation:

CP≧{135−[(35/3)×(3.00−IR)]}×10⁻⁸

wherein CP has units of cm². For example, when the absorbent composite has a 3^{rd} Insult FIFE intake rate (IR) of 3.00 ml/sec, the CP value is desirably equal to or greater than about 135×10⁻⁸ cm². Further, when the absorbent composite has a 3^{rd} Insult FIFE intake rate (IR) of 1.00 ml/sec, the CP value is desirably equal to or greater than about 112×10⁻⁸ cm².

In yet a further embodiment, the absorbent composites of the present invention have a 3^{rd} insult FIFE intake rate (IR) greater than 3.00 ml/sec and less than about 3.70 ml/sec. When the 3^{rd} Insult FIFE intake rate (IR) of the absorbent composite falls within this range, desirably the Composite Permeability (CP) value at full swelling of the absorbent composite is given by the following equation:

CP≧{175−[(400/7)×(3.70−IR)]}×10⁻⁸

wherein CP has units of cm². For example, when the absorbent composite has a 3^{rd} Insult FIFE intake rate (IR) of 3.50 ml/sec, the CP value is desirably equal to or greater than about 164×10⁻⁸ cm². Further, when the absorbent composite has a 3^{rd} Insult FIFE intake rate (IR) of 3.20 mi/sec, the CP value is desirably equal to or greater than about 146×10⁻⁸ cm².

In another embodiment, the absorbent composites of the present invention have a Composite Permeability value/3rd Insult Fluid Intake Flowback Evaluation (FIFE) intake rate relationship represented by the following equations. When the 3^{rd} Insult FIFE intake rate (IR) of the absorbent composite is greater than 0 ml/sec and less than about 3.00 ml/sec, desirably the Composite Permeability (CP) value at full swelling of the absorbent composite is given by the following equation:

CP≧{150−[(35/3)×(3.00−IR)]}×10⁻⁸

wherein CP has units of cm². Further, when the 3^{rd} Insult FIFE intake rate (IR) of the absorbent composite is greater than 3.00 ml/sec and less than about 3.70 ml/sec, desirably the Composite Permeability (CP) value at full swelling of the absorbent composite is given by the following equation:

CP≧{190−[(400/7)×(3.70−IR)]}×10⁻⁸

wherein CP has units of cm². Moreover, when the absorbent composite has a 3^{rd} Insult FIFE intake rate (IR) of greater than about 3.70 ml/sec, the CP value is desirably equal to or greater than about 190×10⁻⁸ cm².

In yet another embodiment, the absorbent composites of the present invention have a Composite Permeability value/3^{rd} Insult Fluid Intake Flowback Evaluation (FIFE) intake rate relationship represented by the following equations. When the 3^{rd} Insult FIFE intake rate (IR) of the absorbent composite is greater than 0 ml/sec and less than about 3.00 ml/sec, desirably the Composite Permeability (CP) value at full swelling of the absorbent composite is given by the following equation:

CP≧{165−[(35/3)×(3.00−IR)]}×10⁻⁸

wherein CP has units of cm². Further, when the 3^{rd} Insult FIFE intake rate (IR) of the absorbent composite is greater than 3.00 ml/sec and less than about 3.70 ml/sec, desirably the Composite Permeability (CP) value at full swelling of the absorbent composite is given by the following equation:

CP≧{205−[(400/7)×(3.70−IR)]}×10⁻⁸

wherein CP has units of cm². Moreover, when the absorbent composite has a 3^{rd} Insult FIFE intake rate (IR) of greater than about 3.70 ml/sec, the CP value is desirably equal to or greater than about 205×10⁻⁸ cm².

The absorbent composites of the present invention may comprise one or more superabsorbent materials. As used herein, the term “superabsorbent material” refers to a water-swellable, water-insoluble organic or inorganic material capable, under the most favorable conditions, of absorbing more than about 15 times its weight in an aqueous solution containing 0.9 weight percent of sodium chloride. Organic materials suitable for use as a superabsorbent material of the present invention may include natural materials such as agar, pectin, guar gum, and the like; as well as synthetic materials, such as synthetic hydrogel polymers. Such hydrogel polymers include, but are not limited to, alkali metal salts of polyacrylic acids, polyacrylamides, polyvinyl alcohol, ethylene maleic anhydride copolymers, polyvinyl ethers, hydroxypropylcellulose, polyvinylmorpholinone; and polymers and copolymers of vinyl sulfonic acid, polyacrylates, polyacrylamides, polyvinylpyrridine, and the like. Other suitable polymers include hydrolyzed acrylonitrile grafted starch, acrylic acid grafted starch, and isobutylene maleic anhydride copolymers and mixtures thereof. The hydrogel polymers are desirably lightly crosslinked to render the material substantially water insoluble. Crosslinking may, for example, be by irradiation or by covalent, ionic, van der Waals, or hydrogen bonding. The superabsorbent materials may be in any form suitable for use in absorbent composites including particles, fibers, flakes, spheres, and the like.

While a wide variety of superabsorbent materials are known, the present invention relates, in one aspect, to the proper selection of one or more superabsorbent materials to allow the formation of absorbent composites having improved composite properties and disposable absorbent garments containing the same. Suitable superabsorbent materials for use in the absorbent composites of the present invention include any superabsorbent material, which enables the formation of an absorbent composite having a Composite Permeability (CP) value at full swelling of greater than about 100×10⁻⁸ cm² and a Composite Permeability/3^{rd} Insult Fluid Intake Flowback Evaluation (FIFE) intake rate relationship as described above. Desirably, the superabsorbent materials used in the absorbent composites of the present invention comprise superabsorbent materials having a high Gel Bed Permeability (GBP) value and a low Absorbency Under Load (AUL) value at 0.6 psi (41,370 dynes/cm²). Such superabsorbent materials are disclosed in co-pending U.S. provisional patent application Serial No. 60/114,432, filed Dec. 31, 1998 as KC No. 14469, entitled “ABSORBENT COMPOSITES COMPRISING SUPERABSORBENT MATERIALS” to Yarbrough et al. and assigned to Kimberly-Clark Worldwide, Inc., the entirety of which is incorporated herein by reference.

In one embodiment of the present invention, the absorbent composites comprise one or more superabsorbent materials in the form of a sodium salt of a cross-linked polyacrylic acid. Such superabsorbent materials include, but are not limited to, Stockhausen W-6543 1 (available from Stockhausen Chemical Company, Inc., Greensboro, N.C.); Dow AFA-173-60B, Dow XU 40671.00, Dow XUS 40665.07, and Dow XUS 40667.01 (all available from The Dow Chemical Company, Midland, Mich.).

In addition to the superabsorbent materials described above, the absorbent composites of the present invention may comprise means to contain the superabsorbent material. Any means capable of containing the above-described superabsorbent materials, which means is further capable of being located in a disposable absorbent garment, is suitable for use in the present invention. Many such containment means are known to those skilled in the art. For example, the containment means may comprise a fibrous matrix such as an air-laid or wet-laid web of cellulosic fibers, a meltblown web of synthetic polymeric fibers, a spunbonded web of synthetic polymeric fibers, a coformed matrix comprising cellulosic fibers and fibers formed from a synthetic polymeric material, air-laid heat-fused webs of synthetic polymeric material, open-celled foams, and the like.

Alternatively, the containment means may comprise two layers of material which are joined together to form a pocket or compartment, more particularly a plurality of pockets, which pocket contains the superabsorbent material. In such a case, at least one of the layers of material should be water-pervious. The second layer of material may be water-pervious or water-impervious. The layers of material may be cloth-like wovens and nonwoven, closed or open-celled foams, perforated films, elastomeric materials, or may be fibrous webs of material. When the containment means comprises layers of material, the material should have a pore structure small enough or tortuous enough to contain the majority of the superabsorbent material. The containment means may also comprise a laminate of two layers of material between which the superabsorbent material is located and contained. Further, the containment means may comprise a support structure, such as a polymeric film, on which the superabsorbent material is affixed. The superabsorbent material may be affixed to one or both sides of the support structure, which may be water-pervious or water-impervious.

Desirably, the absorbent composites of the present invention comprise superabsorbent material in combination with a fibrous matrix containing one or more types of fibrous materials. Suitable fibrous material includes any fibrous material, which enables the formation of an absorbent composite having a Composite Permeability (CP) value at full swelling of greater than about 100×10⁻⁸ cm² and a Composite Permeability/3^{rd} Insult Fluid Intake Flowback Evaluation (FIFE) intake rate relationship as described above. The fibrous material forming the absorbent composites of the present invention may be selected from a variety of materials including natural fibers, synthetic fibers, and combinations thereof. A number of suitable fiber types are disclosed in U.S. Pat. No. 5,601,542, assigned to Kimberly-Clark Corporation, the entirety of which is incorporated herein by reference. The choice of fibers depends upon, for example, the intended end use of the finished absorbent composite. For instance, suitable fibrous materials may include, but are not limited to, natural fibers such as cotton, linen, jute, hemp, wool, wood pulp, etc. Similarly, regenerated cellulosic fibers such as viscose rayon and cuprammonium rayon, modified cellulosic fibers, such as cellulose acetate, or synthetic fibers such as those derived from polyesters, polyamides, polyacrylics, etc., alone or in combination with one another, may likewise be used. Blends of one or more of the above fibers may also be used if so desired.

Fibrous materials may be conveniently characterized by their Water Retention Values (WRV). The test method for determining the WRV of a fiber is described below. For instance, Coosa CR-1654 from Alliance Forest Products (Coosa Pines, Ala.) has a WRV of about 1.0-1.1 g/g; NHB416 from Weyerhaeuser Company (Federal Way, Wash.) has a value of about 0.54 g/g; HBAS from Weyerhaeuser Company has a value of about 0.46 g/g; and synthetic fibers such as those made with polypropylene have a WRV of about zero g/g. High WRV pulps such as CR-1654 are readily available and are widely used in absorbent products. Lower WRV pulps (around 0.5 g/g or less) are less widely used in superabsorbent/fluff pulp mixtures due to their limited compatability with most production technologies. Synthetic fibers such as those made from cellulose acetate, polypropylene, and polyethylene are used in personal care products such as diapers in limited quantity due to their undesirable surface properties. Although the surface properties of these synthetic fibers can be modified by coating with a surfactant, other complications such as surfactant wash-off, may occur.

In one embodiment of the present invention, the absorbent composites comprise a mixture of superabsorbent material and fibrous materials, wherein the WRV of the fibrous material is greater than about 0.2 g/g. Desirably, the WRV of the fibrous material is greater than about 0.35 g/g. More desirably, the WRV of the fibrous material is greater than about 0.5 g/g. Even more desirably, the WRV of the fibrous material is greater than about 0.7 g/g. Most desirably, the WRV of the fibrous material is greater than about 0.9 g/g. Table 6 contains WRV data for a variety of fibers.

TABLE 6
WRV of Various Fibers

| | Type of Fiber | WRV (g/g) |
|---|---|---|
| Regular Coosa Pulp | CR-54* | 1.12 |
| Coosa Pulp After Bivis Refiner | CR-54 Bivis # | 0.94 |
| Crosslinked CR-54 Bivis | CR-28 # | 0.34 |
| Crosslinked CR-54 Bivis | CR-29 # | 0.34 |
| Crosslinked, Debonder Treated | HBAFF** | 0.51 |
| Similar to HBAFF, No Debonder | NHB416** | 0.54 |
| Debonder Treated | HBAS** | 0.46 |
| Crosslinked | Curly Q*** | 0.42 |

*Alliance Forest Products (Coosa Pines, AL)
# KG Modified pulp
**Weyerhaeuser Company (Federal Way, Washington)
***Removed from Proctor & Gamble product

In one embodiment of the present invention, the relative amount of superabsorbent material and fibrous material used to produce the absorbent composites of the present invention may vary depending on the desired properties of the resulting product, and the application of the resulting product. Desirably, the amount of superabsorbent material in the absorbent composite is from about 20 wt % to about 100 wt % and the amount of fibrous material is from about 80 wt % to about 0 wt %, based on the total weight of the absorbent composite. More desirably, the amount of superabsorbent material in the absorbent composite is from about 30 wt % to about 90 wt % and the amount of fibrous material is from about 70 wt % to about 10 wt %, based on the total weight of the absorbent composite. Most desirably, the amount of superabsorbent material in the absorbent composite is from about 40 wt % to about 80 wt % and the amount of fibrous material is from about 60 wt % to about 20 wt %, based on the total weight of the absorbent composite.

In a further embodiment of the present invention, the basis weight of superabsorbent material used to produce the absorbent composites of the present invention may vary depending on the desired properties, such as total composite thickness and basis weight, in the resulting product, and the application of the resulting product. For example, absorbent composites for use in infant diapers may have a lower basis weight and thickness compared to an absorbent composite for an incontinence device. Desirably, the basis weight of superabsorbent material in the absorbent composite is greater than about 80 grams per square meter (gsm). More desirably, the basis weight of superabsorbent material in the absorbent composite is from about 80 gsm to about 800 gsm. More desirably, the basis weight of superabsorbent material in the absorbent composite is from about 120 gsm to about 700 gsm. Most desirably, the basis weight of superabsorbent material in the absorbent composite is from about 150 gsm to about 600 gsm.

The absorbent composites of the present invention may be made by any process known to those of ordinary skill in the art. In one embodiment of the present invention, superabsorbent particles are incorporated into an existing fibrous substrate. Suitable fibrous substrates include, but are not limited to, nonwoven and woven fabrics. In many embodiments, particularly personal care products, preferred substrates are nonwoven fabrics. As used herein, the term “nonwoven fabric” refers to a fabric that has a structure of individual fibers or filaments randomly arranged in a mat-like fashion. Nonwoven fabrics may be made from a variety of processes including, but not limited to, air-laid processes, wet-laid processes, hydroentangling processes, staple fiber carding and bonding, and solution spinning. The superabsorbent material may be applied in the form of a solid particulate material or in situ from a solution. The superabsorbent material may be in any form suitable for use in absorbent composites including particles, fibers, flakes, spheres, and the like.

In a further embodiment of the present invention, the superabsorbent material and fibrous material are simultaneously mixed to form an absorbent composite. Desirably, the composite materials are mixed by an air-forming process known to those of ordinary skill in the art. Air-forming the mixture of fibers and superabsorbent material is intended to encompass both the situation wherein preformed fibers are air-laid with the superabsorbent material, as well as, the situation in which the superabsorbent material is mixed with the fibers as the fibers are being formed, such as through a meltblowing process.

In should be noted that the superabsorbent material may be distributed uniformly within the absorbent composite or may be non-uniformly distributed within the absorbent composite. The superabsorbent material may be distributed throughout the entire absorbent composite or may be distributed within a small, localized area of the absorbent composite.

The absorbent composites of the present invention may be formed from a single layer of absorbent material or multiple layers of absorbent material. In the case of multiple layers, the layers may be positioned in a side-by-side or surface-to-surface relationship and all or a portion of the layers may be bound to adjacent layers. In those instances where the absorbent composite includes multiple layers, the entire thickness of the absorbent composite may contain one or more superabsorbent materials or each individual layer may separately contain some or no superabsorbent materials. Each individual layer may also contain different superabsorbent materials from an adjacent layer. For example, in one embodiment of the present invention, a multiple layer absorbent composite comprises an uppermost absorbent layer (user side) containing one type of superabsorbent material, and a second layer containing a second, different type of superabsorbent material.

The absorbent composites according to the present invention are suited to absorb many fluids including body fluids such as urine, menses, and blood, and are suited for use in absorbent garments such as diapers, adult incontinence products, bed pads, and the like; in catamenial devices such as sanitary napkins, tampons, and the like; and in other absorbent products such as wipes, bibs, wound dressings, food packaging, and the like. Accordingly, in another aspect, the present invention relates to a disposable absorbent garment comprising an absorbent composite as described above. A wide variety of absorbent garments are known to those skilled in the art. The absorbent composites of the present invention can be incorporated into such known absorbent garments. Exemplary absorbent garments are generally described in U.S. Pat. Nos. 4,710,187 issued Dec. 1, 1987, to Boland et al.; 4,762,521 issued Aug. 9, 1988, to Roessler et al.; 4,770,656 issued Sep. 13, 1988, to Proxmire et al.; 4,798,603 issued Jan. 17, 1989; to Meyer et al.; which references are incorporated herein by reference.

As a general rule, the absorbent disposable garments according to the present invention comprise a body-side liner adapted to contact the skin of a wearer, an outer cover superposed in facing relation with the liner, and an absorbent composite, such as those described above, superposed on said outer cover and located between the body-side liner and the outer cover.

Those skilled in the art will readily understand that the absorbent composites of the present invention may be advantageously employed in the preparation of a wide variety of products, including but not limited to, absorbent personal care products designed to be contacted with body fluids. Such products may only comprise a single layer of the absorbent composite or may comprise a combination of elements as described above. Although the absorbent composites of the present invention are particularly suited for personal care products, the absorbent composites may be advantageously employed in a wide variety of consumer products.

### TEST METHODS

The test methods for determining the Water Retention Value (WRV) of fibers and the Composite Permeability Test, the Fluid Intake Flowback Evaluation test, and the Intake/Desorption test of absorbent composites are described below:

Water Retention Value (WRV) Test

The Water Retention Value (WRV) Test determines the water retention of a sample of fibers. In this test, a sample of fibers (0.5 g) is dispersed in at least 100 ml of deionized water and soaked overnight to allow for equilibration. Then, the slurry is poured into a cylinder with an inner diameter of 1.9 inches (4.83 cm). The bottom of the cylinder is covered with a 100 mesh screen so that excess water can drain out of the cylinder. The cylinder is then placed in a standard centrifuge and is spun at 1000 g for 20 minutes. The weight of the pulp is then weighted (W1), dried at 105° C. for two hours, and weighed again (W2). WRV is calculated as (W1-W2)/W2 and given in g/g units.

Composite Permeability Test

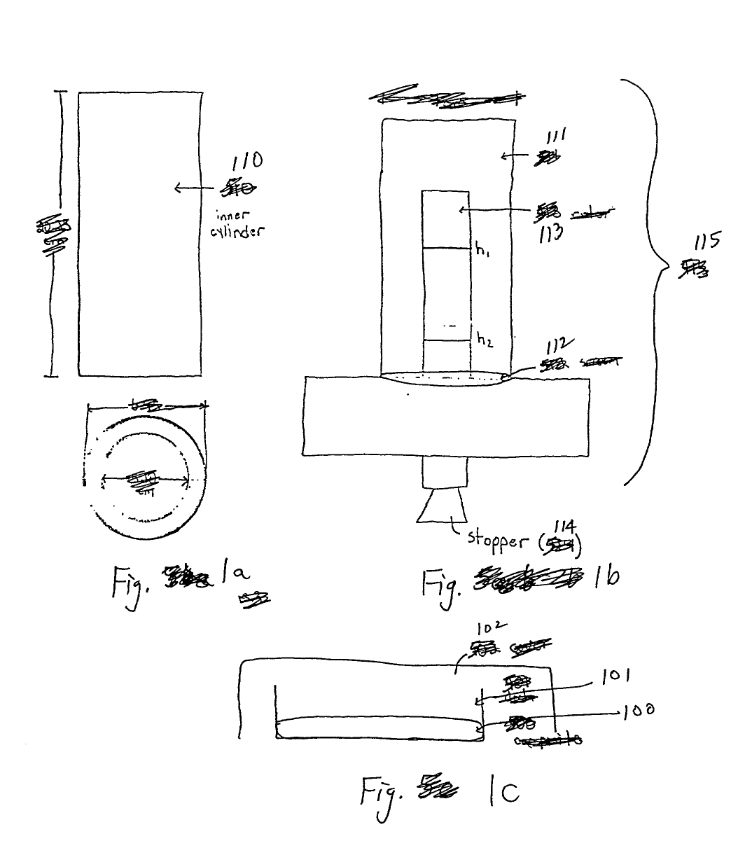

The Composite Permeability test determines the permeability of a composite in cm² by calculating the time for a fluid to flow through a composite. As shown in FIGS. 1a and 1b, the permeability tester consists of two plexiglass or polycarbonate concentric cylinders, wherein one fits inside the other with very little clearance, but still slides freely. The inner cylinder 110 has an outer diameter of 6.9 cm and an inner diameter of 5.10 cm. The outer cylinder/base & stopper assembly 115 has a metal screen 112, on which the test material is placed for testing. This screen is desirably a type 104 stainless steel screen with a hole diameter of 0.156 inches (0.40 cm) and 63% open area, 20 gauge, and 3/16 inch (0.48 cm) center to center spacing. The outer cylinder 111 of the base and stopper assembly has an inner diameter of 7.0 cm and an outer diameter of 7.5; cm. A ruler 113 is on the outside of the outer cylinder 111 with height markings 3 ⅝ inch (9.21 cm) and 1 ⅛ inch (2.86 cm) from the bottom of the screen 112.

An absorbent composite of superabsorbent material and fluff, or fluff alone, is air-formed on tissue to a desired basis weight and density. This composite 300 is die cut to a desired size, desirably, a 6.83 cm (2.69 inch) diameter circle is used. As shown in FIG. 1c, the composite is placed in a dish 101 of approximately the same size (diameter) as the composite 100. This prevents swelling in the radial direction. The sample is saturated using a 0.9% (w/v) aqueous NaCl solution. A cover 102 is placed over the dish and allowed to sit 30 minutes to equilibrate. More solution may be added, if necessary, to fully saturate the sample. One will generally know when the composite is fully saturated when an excess of liquid exists within the dish 101. After a total of 30 minutes, the composite 100 and dish 101 are placed upside down on an absorbent medium such as paper toweling to remove the interstitial liquid. This is done by placing the paper toweling over the dish and composite, and while holding the dish and toweling, flipping it over. This puts the composite in direct contact with the toweling. No pressure is applied during this process.

After the blotting process, a wet bulk thickness of the sample is taken by placing the sample under a thickness gauge with an acrylic platen or the like, which applies approximately 0.05 psi (3,448 dynes/cm²) pressure. The composite is then placed on the inner cylinder 110 and the outer cylinder (permeability tester) 115 is turned upside down over the inner cylinder with the composite. The entire apparatus, which now contains the test composite and the inner cylinder, is flipped back over for the test. This ensures that the composite rests neatly (with least amount of handling) on screen 112 at the bottom of test apparatus 115. The test fluid is poured in the inner cylinder on top of the composite. The fluid should be above the top mark on the ruler (at least 1 inch (2.54 cm)). before starting the test. To initiate the test, the stopper 114 is removed from the bottom of the permeability apparatus 115 and the timer is started when the fluid front reaches the top mark on the ruler (3⅝ inch (9.21 cm) above the screen) and the timer is stopped when the fluid front reaches the bottom mark on the ruler (1⅛ inch (2.86 cm) above the screen). Time in seconds is recorded.

Permeability (K) in cm² is calculated as follows:

K={[(ln (h₁/h₂)*Mu)/(g*Rho)]*WB/t}

where K=composite permeability (cm²); h₁=height of upper marker (cm) [normally 9.21 cm]; h₂=height of lower marker (cm) [normally 2.86 cm]; Mu=liquid viscosity (poise) [normally 0.01 poise]; g=acceleration due to gravity (cm/sec²) [normally 980 cm/sec²]; Rho=liquid density (gm/cm³) [normally 1.0 gm/cm³]; WB=wet bulk of composite (cm); t=time for liquid to move from h₁ to h₂ while flowing through composite (sec).

Fluid Intake Flowback Evaluation test

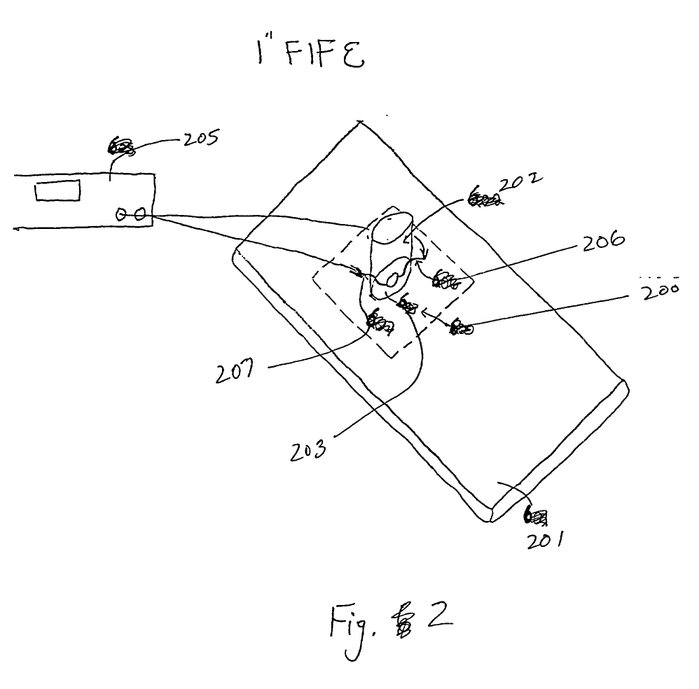

The Fluid Intake Flowback Evaluation (FIFE) test determines the amount of time required for an absorbent composite to intake a preset amount of fluid. A suitable apparatus for performing the FIFE test is shown in FIG. 2.

A composite of superabsorbent and fluff, or fluff only, is air-formed on tissue to a desired basis weight and density. The composite is cut to the desired size, in this case, the composite 200 is cut to a 5 inch (12.70 cm) square. The composite 200 is placed under the FIFE test pad 201. The test pad is a flexible conformable silicon bed that is 10 inches (25.4 cm) by 20 inches (50.8 cm). The silicon pad is constructed using Dow Coming 227 primeness silicon dielectric gel and wrapping it in shrinkable plastic wrapping. This pad is made with a sufficient thickness to produce a pressure of approximately 0.03 psi (2,069 dynes/cm²). The pad contains a plexiglass cylinder 202 with an inner diameter of 5.1 cm and an outer diameter of 6.4 cm and the bottom of the cylinder has a cap 203 with a 1 inch (2.54 cm) circle bore in the center where the test fluid comes in direct contact with the composite 200. The center of the cylinder is located 6.75 inches (17.15 cm) down from the top edge of the silicon pad 201 and is centered from side to side (5 inches (12.70 cm) from the edge). An automated controller 205 can be connected to electrodes 606 and 207 that auto-initiate the test upon the entry of the test fluid. This can eliminate tester variability. The test fluid is desirably a 0.9% (w/v) NaCl solution.

The test is run by placing the composite 200 under the silicon test pad 201. The desired amount of fluid is dispensed from a positive displacement pump. The fluid amount in this case is calculated according to the composition of the composite. For example, the fluid amount for a 400 gsm composite of size 5 inch (12.70 cm) square consisting of 50% superabsorbent and 50% fluff is calculated by assuming the superabsorbent capacity is 30 g/g and the fluff capacity is 6 g/g. The total amount of capacity of the composite in grams is calculated and 25% of this amount is one insult. The fluid is dispensed at a rate of approximately 10 ml/sec. The time in seconds for the fluid to drain from the cylinder 202 is recorded.

After a 15 minute wait, a second insult is done and after another 15 minute wait, the third and final insult is done. The FIFE Intake Rate for each insult is determined by dividing the insult amount in milliliters by the time necessary for the fluid to drain from the cylinder 202 in seconds.

If during the test, leakage of fluid occurs from the top, bottom, or sides of the composite, the amount of leaked fluid should be measured. In this case, the FIFE Intake Rate for each insult is determined by subtracting the leaked fluid amount from the insult fluid amount and then dividing this quantity by the time for the fluid to drain from the cylinder 202 in seconds.

Intake/Desorption Test

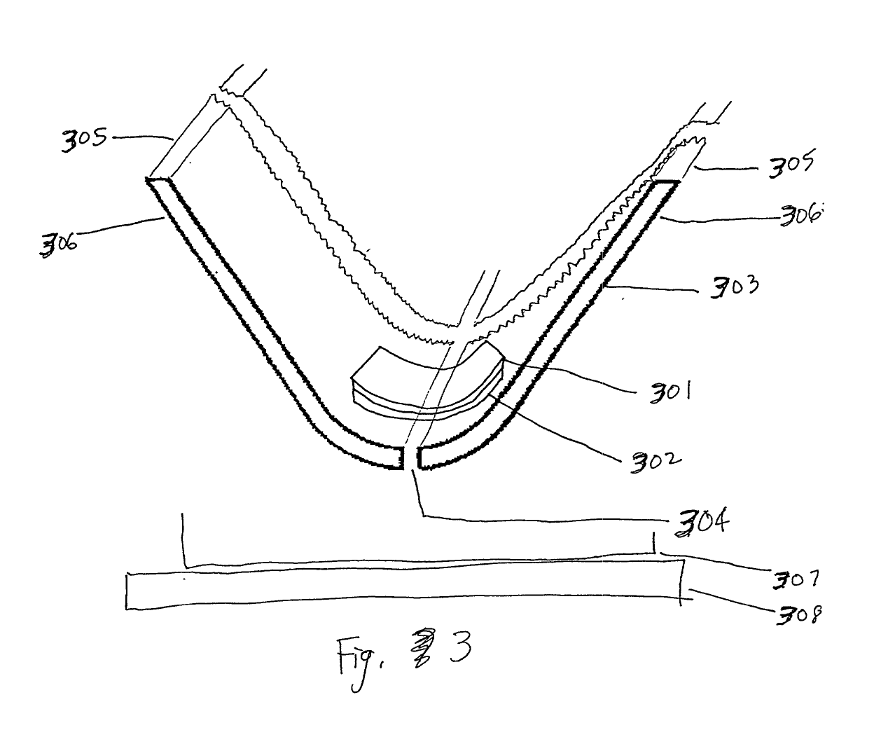

The Intake/Desorption test measures the intake and desorption capability of a material or composite. A suitable apparatus for performing the Intake/Desorption test is shown in FIG. 3.

A composite may consist of superabsorbent material and fluff, or fluff only. In this case, composites consisting of superabsorbent material and fluff were air-formed on tissue to a desired basis weight and density. The composite is then cut to the desired size, in this case, the composite is cut to 2.5 inches (6.35 cm) by 6 inches (15.24 cm). The dry weight of the composite 301 to be tested is recorded. The test composite 301 is placed on a piece of polyethylene film 302 that is the exact size of the test composite 301 and centered in a Plexiglas cradle 303 such that the length of the composite (15.24 cm) is perpendicular to the slot 304 in the bottom of the cradle 303. The cradle 303 has a width of 33 cm. The ends 305 of the cradle 303 are blocked off at a height of 19 cm to form an inner distance of 30.5 cm and an angle between the upper arms of 60 degrees between upper arms 306 of cradle 303. The cradle 303 has a 6.5 mm wide slot 304 at the lowest point running the length of the cradle 303. The slot 304 allows run-off from the test composite 301 to enter tray 307. The amount of run-off is recorded by a balance 308 readable to the nearest 0.01 g. A pre-set amount of liquid is delivered in the center of the test composite 301 at a desired rate. In this case the amount is 100 ml at a rate of 15 ml/sec and ½ inch (1.27 cm) above the sample. The amount of run-off is recorded.

The test composite 301 is immediately removed from the cradle 303 and placed on a 2.5 inches (6.35 cm) by 6 inches (15.24 cm) pre-weighed dry pulp/superabsorbent desorption pad having a density of about 0.20 g/cc in a horizontal position under 0.05 psi pressure for 15 minutes. The superabsorbent material is desirably Favor 880, available from Stockhausen, Inc. (Greensboro, N.C.). The pulp is desirably Coosa 1654, available from Alliance Forest Products (Coosa Pines, Ala.). This pressure is applied by using a Plexiglas plate. After the 15 minutes, the desorption pad weight is recorded and the test composite 301 is placed back in the cradle 303 and a second insult of 100 ml is done. After the amount of run-off is recorded, the test composite 301 is once again placed on a pre-weighed dry desorption pad under 0.05 psi (dynes/cm²) load for 15 minutes. After 15 minutes, a weight of the desorption pad is recorded. The composite 301 is placed back in the cradle 303 for a third insult. The amount of run-off is recorded and the test composite 301 is placed on a dry pre-weighed desorption pad under 0.05 psi pressure for 15 minutes. The amount of fluid picked up in g/g for each insult is calculated by subtracting the run-off from 100 ml and dividing by the dry weight of the test composite 301. A particularly useful measure of the ability of a composite to exhibit superior fluid intake of multiple insults over the life of the composite is to divide the 3^{rd} insult pickup value by the 1^{st} insult pickup value.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims,

### EXAMPLE 1

Testing of Absorbent Composites for Composite Permeability, 3^{rd} FIFE Intake Rate, and Intake/Desorption 3^{rd}/1^{st} Pickup

Absorbent composites were prepared and evaluated for one or more of the following: Composite Permeability at full swelling, 3^{rd} FIFE Intake Rate, and Intake/Desorption 3^{rd}/1^{st} Pickup. Each composite was formed from superabsorbent material combined with fluffed pulp fibers (Coosa River CR-1654; available from Alliance Forest Products (Coosa Pines, Ala.). The materials were formed into webs using conventional air-forming equipment. The weight percent of superabsorbent material and the basis weight of superabsorbent material was varied as shown in Table 7.

TABLE 7
Nonwoven Absorbent Composites of Superabsorbent
Material and Wood Pulp Fibers

| | | | SAM | |
|---|---|---|---|---|
| | SAM | | Concentra- | SAM Basis |
| | Desig- | | tion | Weight |
| Sample | nation | SAM | (mass %) | (gsm) |
| 1 | S1 | Stockhausen W- | 50 | 200 |
| | | 65431 | | |
| 2 | D2 | Dow AFA-173-60B | 50 | 200 |
| 3 | D4 | Dow XU 40671.00 | 50 | 200 |
| 4 | D3 | Dow XUS 40665.07 | 50 | 200 |
| 6 | D6 | Dow XUS | 50 | 200 |
| | | 40667.01 | | |
| 5 | D7 | Dow XU40669 | 50 | 200 |
| C-2 | — | Stockhausen 880 | 50 | 200 |
| | | (600-850 micron) | | |
| 12 | S2 | Stockhausen W- | 50 | 200 |
| | | 65406 | | |
| 13 | S3 | Stockhausen W- | 50 | 200 |
| | | 77553 | | |
| C-6 | — | Stockhausen 880 | 50 | 200 |
| C-7 | — | Dow 2035 | 50 | 200 |
| 7 | D3 | Dow XUS 40665.07 | 50 | 250 |
| 8 | D3 | Dow XUS 40665.07 | 50 | 150 |
| 14 | D3 | Dow XUS 40665.07 | 50 | 100 |
| 9 | D3 | Dow XUS 40665.07 | 60 | 240 |
| 10 | D3 | Dow XUS 40665.07 | 40 | 160 |
| 11 | D3 | Dow XUS 40665.07 | 30 | 120 |
| C-10 | — | Stockhausen 880 | 50 | 250 |
| C-11 | — | Stockhausen 880 | 50 | 150 |
| C-12 | — | Stockhausen 880 | 50 | 100 |
| C-13 | — | Stockhausen 880 | 60 | 240 |
| C-14 | — | Stockhausen 880 | 40 | 160 |
| C-15 | — | Stockhausen 880 | 30 | 120 |

The composites identified as samples 1 to 14 and comparative examples C-2, C-6 to C-7, and C-10 to C-15 were evaluated for one or more of the following: composite permeability, 3^{rd} FIFE Intake Rate, and Intake/Desorption 3^{rd}/1^{st} Pickup as described above. The results of these tests are shown in Table 8.

TABLE 8
Testing for Composite Permeability, 3^{rd} FIFE Intake
Rate, and Intake/Desorption 3^{rd}/1^{st} Pickup

| | Composite | 3^{rd} FIFE Intake | |
|---|---|---|---|
| | Permeability | Rate | Intake/Desorption 3^{rd}/1^{st} |
| Sample | (×10⁻⁸ cm²) | (ml/sec) | Pickup |
| 1 | 191 | 3.2 | 1 |
| 2 | 177 | 3.1 | 1.18 |
| 3 | 192 | 3.4 | 1.35 |
| 4 | 202 | 3.1 | 1.52 |
| 6 | 168 | 3.0 | 1.22 |
| 7 | — | 3.5 | — |
| 8 | — | 5.5 | — |
| 9 | 159 | 3.0 | — |
| 10 | 255 | 5.7 | — |
| 5 | 163 | 2.7 | 0.92 |
| C-2 | 110 | 2.5 | 0.99 |
| 12 | 198 | 2.0 | 0.86 |
| 13 | 152 | 1.7 | 0.98 |
| C-6 | 112 | 2.2 | 0.90 |
| C-7 | 61 | 1.6 | 0.92 |
| 14 | — | 4.4 | — |
| 11 | 226 | 6.6 | — |
| C-10 | — | 2.5 | — |
| C-11 | — | 3.3 | — |
| C-12 | — | 4.4 | — |
| C-13 | 63 | 2.0 | — |
| C-14 | 172 | 4.0 | — |
| C-15 | 161 | 6.6 | — |

As can be seen when examining the above data, the absorbent composites having improved intake performance may be formed.

The above disclosed examples are preferred embodiments and are not intended to limit the scope of the present invention in any way. Various modifications and other embodiments and uses of the disclosed superabsorbent polymers, apparent to those of ordinary skill in the art, are also considered to be within the scope of the present invention.

What is claimed is:

1. An absorbent composite comprising about 20 weight percent to 100 weight percent, based on the total weight of the composite, of superabsorbent material; wherein the absorbent composite has a Composite Permeability (CP) value at full swelling and a 3^{rd} Insult Fluid Intake Flowback Evaluation (FIFE) intake rate (IR); and wherein the CP value and the IR value satisfy the following conditions:
when the IR value of the absorbent composite is greater than 0 ml/sec and less than about 3.00 ml/sec, the CP value is given by the following equation:
CP≧{135−[(35/3)×(3.00−IR)]}×10⁻⁸;
when the IR value of the absorbent composite is greater than about 3.00 ml/sec and less than about 3.70 ml/sec, the CP value is given by the following equation:
CP≧{175−[(400/7)×(3.70−IR)]}×10⁻⁸;
and
when the IR value of the absorbent composite is greater than about 3.70 ml/sec, the CP value is greater than or equal to 175×10⁻⁸, wherein CP has units of cm².

2. The absorbent composite of claim 1, wherein the CP value and the IR value satisfy the following conditions:
when the IR value of the absorbent composite is greater than 0 ml/sec and less than about 3.00 ml/sec, the CP value is given by the following equation:
CP≧{150−[(35/3)×(3.00−IR)]}×10⁻⁸;
when the IR value of the absorbent composite is greater than about 3.00 ml/sec and less than about 3.70 ml/sec, the CP value is given by the following equation:
CP≧{190−[(400/7)×(3.70−IR)]}×10⁻⁸;
and
when the IR value of the absorbent composite is greater than about 3.70 ml/sec, the CP value is greater than or equal to 190×10⁻⁸, wherein CP has units of cm².

3. The absorbent composite of claim 2, wherein the CP value and the IR value satisfy the following conditions:
when the IR value of the absorbent composite is greater than 0 ml/sec and less than about 3.00 ml/sec, the CP value is given by the following equation:
CP≧{165−[(35/3)×(3.00−IR)]}×10⁻⁸;
when the IR value of the absorbent composite is greater than about 3.00 ml/sec and less than about 3.70 ml/sec, the CP value is given by the following equation:
CP≧{205−[(400/7)×(3.70−IR)]}×10⁻⁸;
and
when the IR value of the absorbent composite is greater than about 3.70 ml/sec, the CP value is greater than or equal to 205×10⁻⁸, wherein CP has units of cm².

4. The absorbent composite of claim 1, wherein the absorbent composite has a CP value at full swelling of greater than about 175×10⁻⁸ cm².

5. The absorbent composite of claim 1, wherein the absorbent composite has a CP value at full swelling of greater than about 190×10⁻⁸ cm².

6. The absorbent composite of claim 1, wherein the absorbent composite has a CP value at full swelling of greater than about 205×10⁻⁸ cm².

7. The absorbent composite of claim 1, wherein the absorbent composite has a CP value at full swelling of greater than about 225×10⁻⁸ cm².

8. The absorbent composite of claim 1, wherein the absorbent composite has a 3^{rd} Insult FIFE intake rate of about 2.00 ml/sec and a CP value at full swelling of greater than about 125×10⁻⁸ cm².

9. The absorbent composite of claim 1, wherein the absorbent composite has a 3^{rd} Insult FIFE intake rate of greater than about 0.1 ml/sec and a CP value at full swelling of greater than about 175×10⁻⁸ cm².

10. The absorbent composite of claim 1, wherein the absorbent composite has a 3^{rd} Insult FIFE intake rate of greater than about 2.50 ml/sec and a CP value at full swelling of greater than about 175×10⁻⁸ cm².

11. The absorbent composite of claim 1, wherein the absorbent composite has a 3^{rd} Insult FIFE intake rate of greater than about 3.00 ml/sec and a CP value at full swelling of greater than about 175×10⁻⁸ cm².

12. The absorbent composite of claim 1, wherein the absorbent composite further comprises from about 80 to about 0 weight percent fibers.

13. The absorbent composite of claim 12, wherein the absorbent composite comprises from about 30 to about 90 weight percent superabsorbent material and from about 70 to about 10 weight percent fibers.

14. The absorbent composite of claim 13, wherein the absorbent composite comprises from about 40 to about 80 weight percent superabsorbent material and from about 60 to about 20 weight percent fibers.

15. The absorbent composite of claim 12, wherein the fibers have a water retention value (WRV) greater than about 0.2 g/g.

16. The absorbent composite of claim 15, wherein the fibers have a water retention value (WRV) greater than about 0.5 g/g.

17. The absorbent composite of claim 16, wherein the fibers have a water retention value (WRV) greater than about 0.7 g/g.

18. The absorbent composite of claim 17, wherein the fibers have a water retention value (WRV) greater than about 0.9 g/g.

19. The absorbent composite of claim 1, wherein the absorbent composite has a basis weight of superabsorbent material of greater than about 80 grams per square meter.

20. The absorbent composite of claim 19, wherein the absorbent composite has a basis weight of superabsorbent material of from about 80 grams per square meter to about 800 grams per square meter.

21. The absorbent composite of claim 20, wherein the absorbent composite has a basis weight of superabsorbent material of from about 120 grams per square meter to about 700 grams per square meter.

22. The absorbent composite of claim 21, wherein the absorbent composite has a basis weight of superabsorbent material of from about 150 grams per square meter to about 600 grams per square meter.

23. The absorbent composite of claim 1, wherein the superabsorbent material comprises a sodium polyacrylate.

24. An absorbent composite comprising from about 20 to about 100 weight percent superabsorbent material and from about 80 to about 0 weight percent fibers, wherein the composite has a Composite Permeability (CP) value at full swelling of greater than or equal to 175×10⁻⁸ cm².

25. The absorbent composite of claim 24, wherein the fibers have a water retention value (WRV) greater than about 0.2 g/g.

26. The absorbent composite of claim 25, wherein the fibers have a water retention value (WRV) greater than about 0.5 g/g.

27. The absorbent composite of claim 26, wherein the fibers have a water retention value (WRV) greater than about 0.7 g/g.

28. The absorbent composite of claim 27, wherein the fibers have a water retention value (WRV) greater than about 0.9 g/g.

29. A method of making an absorbent composite, said method comprising:
forming an absorbent composite comprising about 20 weight percent to 100 weight percent, based on the total weight of the composite, of superabsorbent material; wherein the absorbent composite has a Composite Permeability (CP) value at full swelling and a 3^{rd} Insult Fluid Intake Flowback Evaluation (FIFE) intake rate (IR); and wherein the CP value and the IR value satisfy the following conditions:
when the IR value of the absorbent composite is greater than 0 ml/sec and less than about 3.00 ml/sec, the CP value is given by the following equation:
CP≧{135−[(35/3)×(3.00−IR)]}×10⁻⁸;
when the IR value of the absorbent composite is greater than about 3.00 ml/sec and less than about 3.70 ml/sec, the CP value is given by the following equation:
CP≧{175−[(400/7)×(3.70−IR)]}×10⁻⁸;
and
when the IR value of the absorbent composite is greater than about 3.70 ml/sec, the CP value is greater than or equal to 175×10⁻⁸, wherein CP has units of cm².

30. The method of claim 29, wherein the CP value and the IR value satisfy the following conditions:
when the IR value of the absorbent composite is greater than 0 ml/sec and less than about 3.00 ml/sec, the CP value is given by the following equation:
CP≧{150−[(35/3)×(3.00−IR)]}×10⁻⁸;
when the IR value of the absorbent composite is greater than about 3.00 ml/sec and less than about 3.70 ml/sec, the CP value is given by the following equation:
CP≧{190−[(400/7)×(3.70−IR)]}×10⁻⁸;
and
when the IR value of the absorbent composite is greater than about 3.70 ml/sec, the CP value is greater than or equal to 190×10⁻⁸, wherein CP has units of cm².

31. The method of claim 30, wherein the CP value and the IR value satisfy the following conditions:
when the IR value of the absorbent composite is greater than 0 ml/sec and less than about 3.00 ml/sec, the CP value is given by the following equation:
CP≧{165−[(35/3)×(3.00−IR)]}×10⁻⁸;
when the IR value of the absorbent composite is greater than about 3.00 ml/sec and less than about 3.70 mil/sec, the CP value is given by the following equation:
CP≧{205−[(400/7)×(3.70−IR)]}×10⁻⁸;
and
when the IR value of the absorbent composite is greater than about 3.70 ml/sec, the CP value is greater than or equal to 205×10⁻⁸, wherein CP has units of cm².

32. The method of claim 29, wherein the absorbent composite has a CP value at full swelling of greater than about 175×10⁻⁸ cm².

33. The method of claim 29, wherein the absorbent composite is formed by an air-forming step.

34. The method of claim 29, wherein the absorbent composite further comprises from about 80 to about 0 weight percent fibers.

35. The method of claim 29, wherein the absorbent composite has a basis weight of superabsorbent material of greater than about 80 grams per square meter.

36. The method of claim 35, wherein the absorbent composite has a basis weight of superabsorbent material of from about 80 grams per square meter to about 800 grams per square meter.

37. The method of claim 29, wherein the superabsorbent material comprises a sodium polyacrylate.

38. A disposable garment comprising the absorbent composite of claim 1.

39. A disposable garment comprising at least one absorbent composite, wherein the at least one absorbent composite comprises about 20 weight percent to 100 weight percent, based on the total weight of the composite, of superabsorbent material; wherein the absorbent composite has a Composite Permeability (CP) value at full swelling and a 3^{rd} Insult Fluid Intake Flowback Evaluation (FIFE) intake rate (IR); and wherein the CP value and the IR value satisfy the following conditions:
when the IR value of the absorbent composite is greater than 0 ml/sec and less than about 3.00 ml/sec, the CP value is given by the following equation:
CP≧{135−[(35/3)×(3.00−IR)]}×10⁻⁸;
when the IR value of the absorbent composite is greater than about 3.00 ml/sec and less than about 3.70 ml/sec, the CP value is given by the following equation:
CP≧{175−[(400/7)×(3.70−IR)]}×10⁻⁸;
and
when the IR value of the absorbent composite is greater than about 3.70 ml/sec, the CP value is greater than or equal to 175×10⁻⁸, wherein CP has units of cm².

40. The disposible garment of claim 39, wherein the CP value and the IR value satisfy the following conditions:
when the IR value of the absorbent composite is greater than 0 ml/sec and less than about 3.00 ml/sec, the CP value is given by the following equation:
CP≧{150−[(35/3)×(3.00−IR)]}×10⁻⁸;
when the IR value of the absorbent composite is greater than about 3.00 ml/sec and less than about 3.70 ml/sec, the CP value is given by the following equation:
CP≧{190−[(400/7)×(3.70−IR)]}×10⁻⁸;
and
when the IR value of the absorbent composite is greater than about 3.70 ml/sec, the CP value is greater than or equal to 190×10⁻⁸, wherein CP has units of cm².

41. The disposible garment of claim 40, wherein the CP value and the IR value satisfy the following conditions:
when the IR value of the absorbent composite is greater than 0 ml/sec and less than about 3.00 ml/sec, the CP value is given by the following equation:
CP≧{165−[(35/3)×(3.00−IR)]}×10⁻⁸;
when the IR value of the absorbent composite is greater than about 3.00 ml/sec and less than about 3.70 ml/sec, the CP value is given by the following equation:
CP≧{205−[(400/7)×(3.70−IR)]}×10⁻⁸;
and
when the IR value of the absorbent composite is greater than about 3.70 ml/sec, the CP value is greater than or equal to 205×10⁻⁸, wherein CP has units of cm².
